(19) United States
(12) Patent Application Publication
(10) Pub. No.: US 2015/0124939 A1
(43) Pub. Date: May 7, 2015

(54) RAIL SYSTEM INCLUDING ADJUSTABLE DETECTOR

(71) Applicant: MEDIEN INTERNATIONAL CO.,LTD, Gyeonggi-do (KR)

(72) Inventor: Pan Soon AHN, Gyeonggi-do (KR)

(21) Appl. No.: 14/329,761

(22) Filed: Jul. 11, 2014

(30) Foreign Application Priority Data
Nov. 1, 2013 (KR) 10-2013-0131936

(51) Int. Cl. A61B 6/00

(52) U.S. Cl. CPC A61B 6/447; A61B 6/588
USPC 378/167

(57) ABSTRACT

A rail system including an adjustable detector. A rail unit is elongated in a longitudinal direction, and includes first and second rails which are spaced apart from each other in a horizontal direction and are fixed to the ground. An X-ray tube stand is movable in a lateral direction with respect to the rail unit, and is provided therein with a vertically-movable tube arm and an X-ray generator which is rotatably disposed on one end of the tube arm. A C-shaped X-ray photography table is spaced apart from the rail unit. An X-ray sensor stand is movable in a lateral direction with respect to the rail unit, and moves an X-ray detector disposed therein into a hollow space in the lower part of the X-ray photography table such that the centers of the X-ray detector and the X-ray photography table are positioned at the same point.

### BACKGROUND OF THE INVENTION

1. Field of the Invention

The present invention relates to a surgical X-ray diagnostic apparatus, and more particularly, to a rail system including an adjustable detector with which one detector (or cassette tray) can function as two detectors (or cassette trays).

2. Description of the Related Art

In general, a surgical X-ray diagnostic apparatus can acquire an image of an affected part of a patient by X-ray photography using one detector (or cassette tray) or two detectors (or cassette trays) and diagnose the acquired image.

The surgical X-ray diagnostic apparatus using two detectors (or cassette trays), which hereinafter will be referred to as the “2D system” including two cassette tray mounting systems, is convenient since it photographs the entire parts of a patient using the two detectors (or cassette trays). However, since the two detectors (or cassette trays) must be concurrently purchased and mounted, the 2D system is expensive. It is therefore difficult for small and medium sized hospitals and clinics to purchase the 2D system, and is generally only an option for large general hospitals with sufficient resources.

Therefore, many hospitals and clinics use a surgical diagnostic apparatus using one detector (or cassette tray), which hereinafter will be referred to as the “1D system” including one cassette tray mounting system. Although the 1D system can be purchased at a cheaper price than the 2D system, the 1D system has several drawbacks in relation to system operation.

Among these drawbacks, first, it takes a long time to reposition when photographing a patent. Second, a mobile table having wheels (hereinafter referred to as a mobile table) must be used when photographing a patient positioned on the table. However, there are some difficulties depending on the weight of the patient when moving the patient on the mobile table. In particular, it is difficult for female radiologists to change the position of a patient resting on the table. Third, when no patients are to be photographed on the table, there are temporal/spatial constraints in moving the mobile table in one direction.

In order to complement for these drawbacks of the 1D system of the related art, devised was a one-legged table (see FIG. 1B; hereinafter referred to as a one-legged table) which is opened in one direction such that a detector can be positioned below the table when the detector is converted from a chest photographing mode into a table photographing mode. However, the one-legged table is also expensive since it must be made of a carbon plate, which is an expensive material.

In addition, when a patient or a heavy person to be photographed is positioned on an end portion of the one-legged table where no lower leg is provided, the end portion may be deformed, which is a structural problem of the one-legged table.

According to the related art, although the 2D system was developed in order to complement for the drawbacks of the 1D system, the 1D system is used in most hospitals or clinics regardless of its drawbacks since the 2D system is expensive. Therefore, there is required a system that can solve the inconvenience problems of the related art.

The information disclosed in the Background of the Invention section is only for the enhancement of understanding of the background of the invention, and should not be taken as an acknowledgment or any form of suggestion that this information forms a prior art that would already be known to a person skilled in the art.

### RELATED ART DOCUMENT

Patent Document 1: Korean Patent No. 10-0946999 (entitled “RAIL SYSTEM AND X-RAY PHOTOGRAPHYING APPARATUS USING THE SAME”)

### SUMMARY OF THE INVENTION

Accordingly, the present invention has been made keeping in mind the above problems occurring in the related art, and the present invention is intended to provide an improved 1D system that has advantages of a 2D system by complementing for drawbacks of the conventional 1D system that stay unsolved in the related art. The present invention is therefore intended to reduce a time required for changing a photographing position, improve user convenience, and obtain a competitive price so that the improved 1D system can be widely used by a number of hospitals, medical centers and clinics.

In order to achieve the above object, according to one aspect of the present invention, there is provided a rail system including an adjustable detector. The rail system includes a rail unit elongated in a longitudinal direction. The rail unit includes first and second rails which are spaced apart from each other in a horizontal direction and are fixed to the ground. The rail system also includes an X-ray tube stand movable in a lateral direction with respect to the rail unit. The X-ray tube stand is provided therein with a tube arm which is movable in the vertical direction and an X-ray generator which is rotatably disposed on one end of the tube arm. The rail system also includes a C-shaped X-ray photography table spaced apart from the rail unit and an X-ray sensor stand movable in a lateral direction with respect to the rail unit. The X-ray sensor stand includes an X-ray detector disposed therein, and moves the X-ray detector into a hollow space in the lower part of the X-ray photography table such that the center of the X-ray detector and the center of the X-ray photography table are positioned at the same point.

According to an embodiment of the present invention, the rail system may further include an auxiliary photography table oriented perpendicular to the X-ray photography table. The auxiliary photography table is attached to and detached from the X-ray photography table by sliding, and is used when photographing a Decubitus posture of a patient.

The rail unit includes: a fixed first rail elongated in the longitudinal direction; a fixed second rail elongated in the longitudinal direction, the second rail being spaced apart from the first rail in a horizontal direction; a fixed first rack gear disposed over the entire length of the first rail; and a fixed second rack gear disposed over the entire length of the second rail.

The X-ray sensor stand may include: a housing having a guide rail therein; a vertical movement section which moves in the vertical direction along the guide rail; and a rotation driving section connected with the vertical movement section. The rotation driving section rotates the X-ray detector. The X-ray sensor stand may also include: a detector arm connected with the rotation driving section, the length of the detector arm being adjustable in a horizontal direction using a potential meter; and the X-ray detector having a guide rail on one surface thereof such that the detector arm is movable in the forward-backward direction.

The detector arm may include: a rack gear, with one end thereof being connected with the rotation driving section; and a pulley disposed on one end of the rack gear such that the rack gear is movable in the forward-backward direction along the guide rail.

The X-ray detector may include: at least one pinion gear engaging with the rack gear of the detector arm; and a potential meter which rotates the pinion gear such that the rack gear moves along the guide rail in a lateral direction.

The rail system may further include a sound unit which generates an alarm sound when the center of the X-ray detector and the center of the X-ray photography table are positioned at the same point.

The X-ray tube stand may further include a distance measuring sensor which detects an interference between the X-ray tube stand and the X-ray sensor stand.

The X-ray photography table may include an upper plate which is expandable in a horizontal direction, whereby the center of the upper plate is freely movable to the center of the X-ray detector.

The rail system including an adjustable detector according to the present invention can overcome the problem of requiring a long time to change the position of a patient for photographing. It is also possible to overcome the problem in that it is difficult to move the mobile table having wheels due to the weight of a patient when changing the photographing position of the patient. The invention has advantages in that the positions of the detector stand and the photographing position as well as the X-ray photographing position can be changed depending on the patient and the length of time required for changing the photographing position can be reduced.

### BRIEF DESCRIPTION OF THE DRAWINGS

The above and other objects, features and advantages of the present invention will be more clearly understood from the following detailed description when taken in conjunction with the accompanying drawings, in which:

FIG. 1A is an exemplary view showing an example of a conventional 1D system;

FIG. 1B is an exemplary view showing an example of a conventional one-legged table;

FIG. 1C is an exemplary view showing an example of a conventional 2D system;

FIG. 2 is an exemplary view showing an example of chest photographing using a rail system including an adjustable detector according to an exemplary embodiment of the present invention;

FIG. 3 is an exemplary view showing another example of FIG. 2;

FIG. 4 is an exemplary view showing a further example of FIG. 3;

FIG. 5A and FIG. 5B are views sequentially showing an operation in which an X-ray detector is introduced into an X-ray photography table;

FIG. 6 is a view showing the state in which the X-ray detector shown in FIG. 5 has moved upwards;

FIG. 7 is a view showing the state in which the X-ray detector has been introduced into the X-ray photography table;

FIG. 8 and FIG. 9 are top plan views sequentially showing the operating process of the X-ray detector and an X-ray generator during Decubitus photographing using the rail system including an adjustable detector according to an exemplary embodiment of the present invention;

FIG. 10 is a side elevation view of FIG. 9;

FIG. 11 is a front elevation view of FIG. 9;

FIG. 12 is an enlarged view of the part A shown in FIG. 10;

FIG. 13 is an exemplary view illustrating that the X-ray detector shown in FIG. 7 expands in a horizontal direction;

FIG. 14 is an exemplary view viewed from the opposite side to FIG. 13;

FIG. 15 is an enlarged view of the X-ray detector;

FIG. 16A to FIG. 16C are views sequentially illustrating the operating process of the X-ray detector which is introduced into a hollow space inside the X-ray photography table; and

FIG. 17A and FIG. 17B are side exemplary views illustrating the process in which the upper section of the X-ray photography table is moved in a horizontal direction.

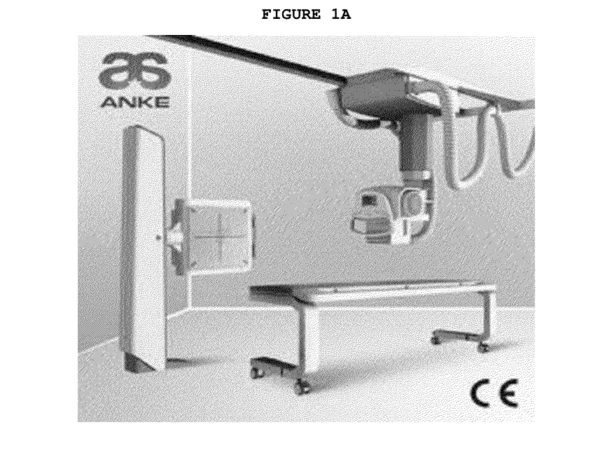

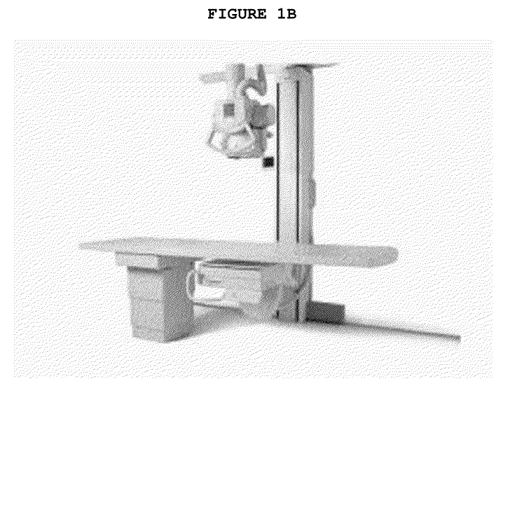

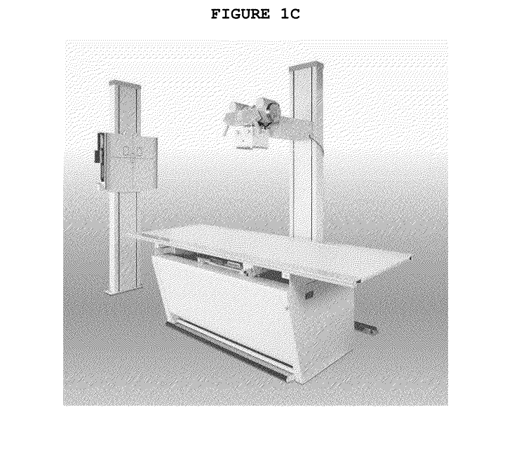

### DETAILED DESCRIPTION OF THE INVENTION

Exemplary embodiments of the present invention will be described more fully hereinafter with reference to the accompanying drawings. In the following description of the present invention, detailed descriptions of known functions and components incorporated herein will be omitted when it may make the subject matter of the present invention unclear.

Reference will now be made in detail to various embodiments of the present invention, specific examples of which are illustrated in the accompanying drawings and described below, since the embodiments of the present invention can be variously modified in many different forms. While the present invention will be described in conjunction with exemplary embodiments thereof, it is to be understood that the present description is not intended to limit the present invention to those exemplary embodiments. On the contrary, the present invention is intended to cover not only the exemplary embodiments, but also various alternatives, modifications, equivalents and other embodiments that may be included within the spirit and scope of the present invention as defined by the appended claims.

It will be understood that when an element is referred to as being “coupled” or “connected” to another element, it can be directly coupled or connected to the other element or intervening elements may be present therebetween. In contrast, it should be understood that when an element is referred to as being “directly coupled” or “directly connected” to another element, there are no intervening elements present. Other expressions that explain the relationship between elements, such as “between,” “directly between,” “adjacent to,” or “directly adjacent to,” should be construed in the same way.

The terminology used herein is for the purpose of describing particular embodiments only and is not intended to be limiting. As used herein, the singular forms “a,” “an” and “the” are intended to include the plural forms as well, unless the context clearly indicates otherwise. It will be further understood that the terms “comprise”, “include”, “have”, etc. when used in this specification, specify the presence of stated features, integers, steps, operations, elements, components, and/or combinations of them but do not preclude the presence or addition of one or more other features, integers, steps, operations, elements, components, and/or combinations thereof.

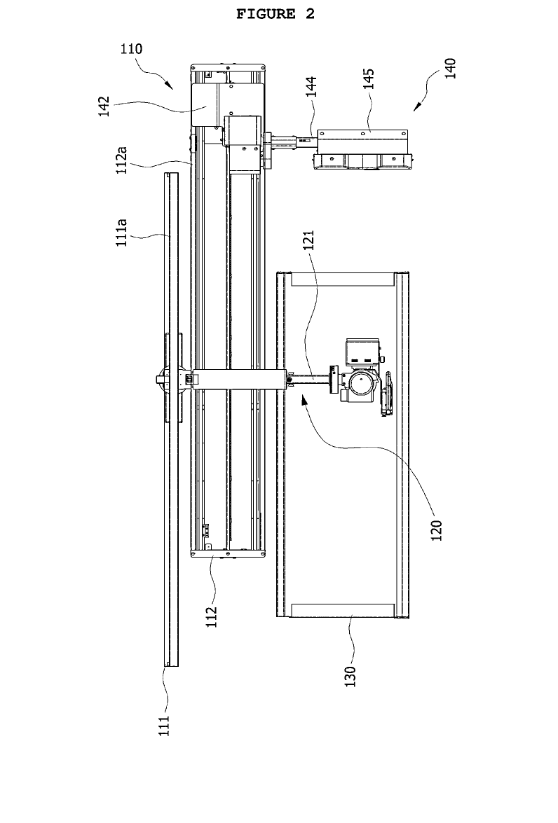

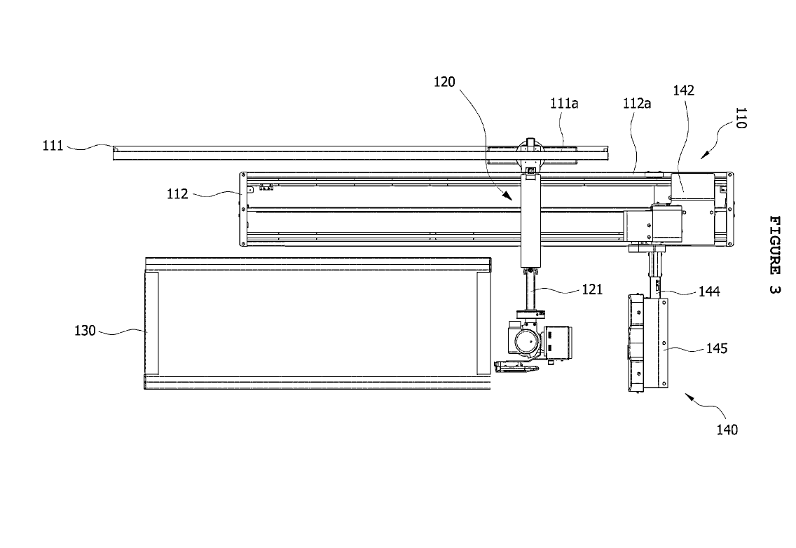

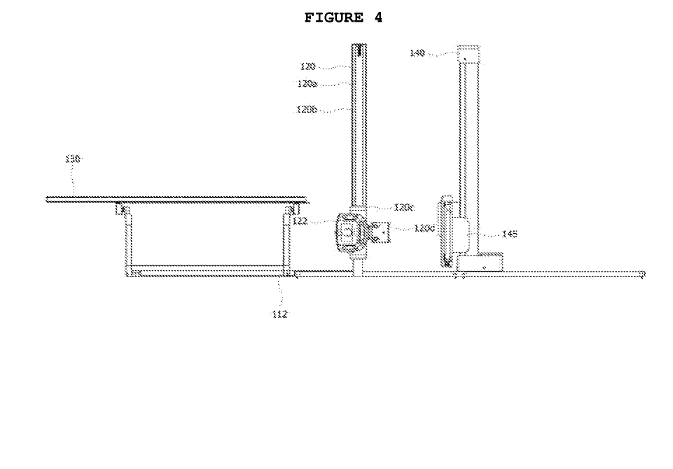

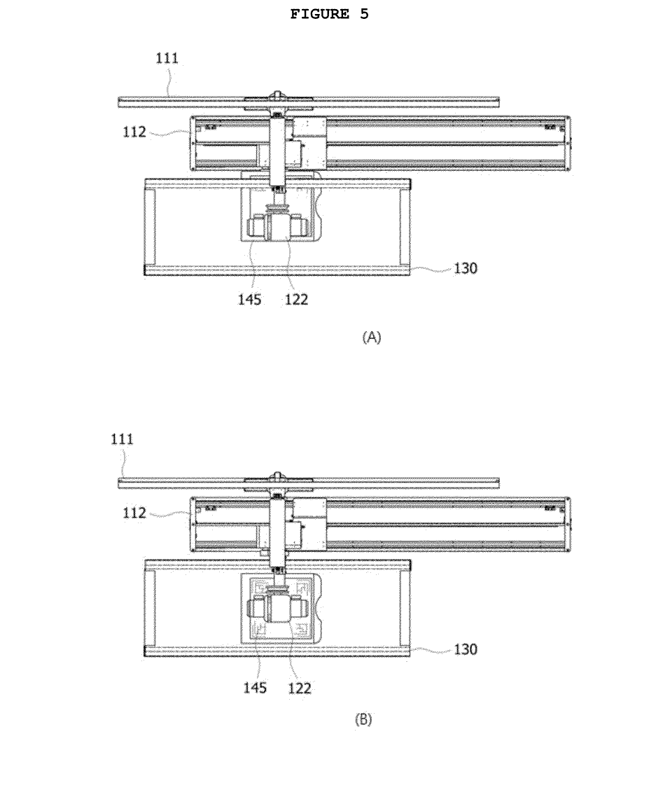

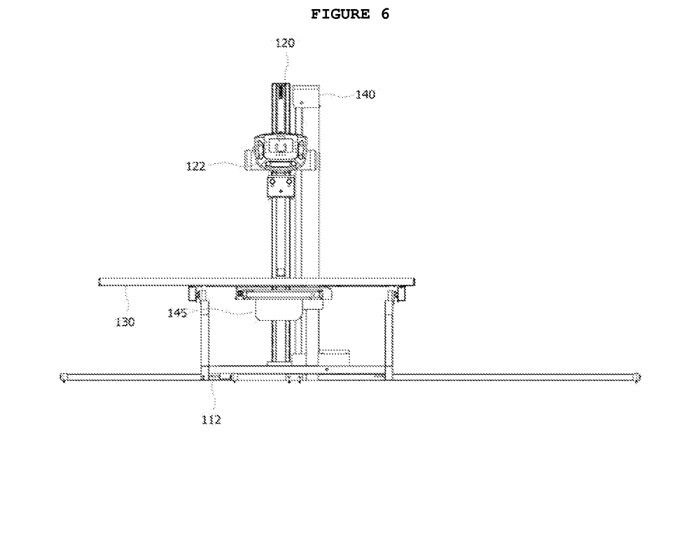

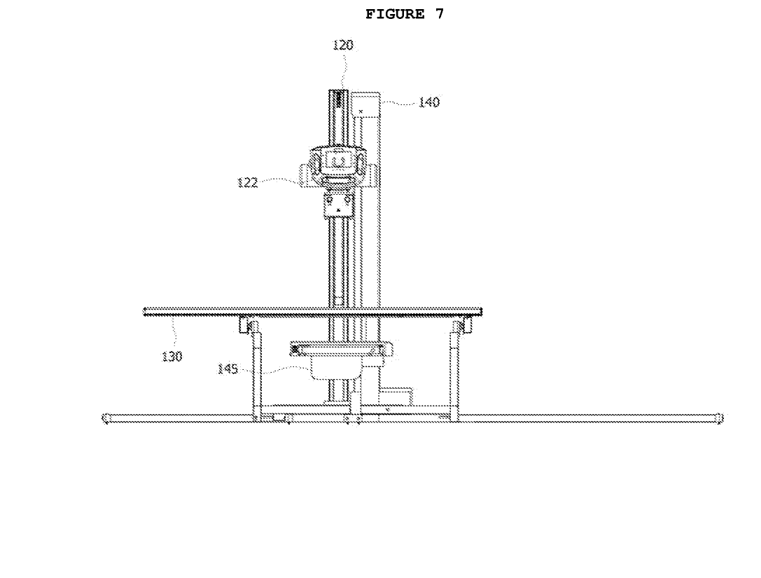

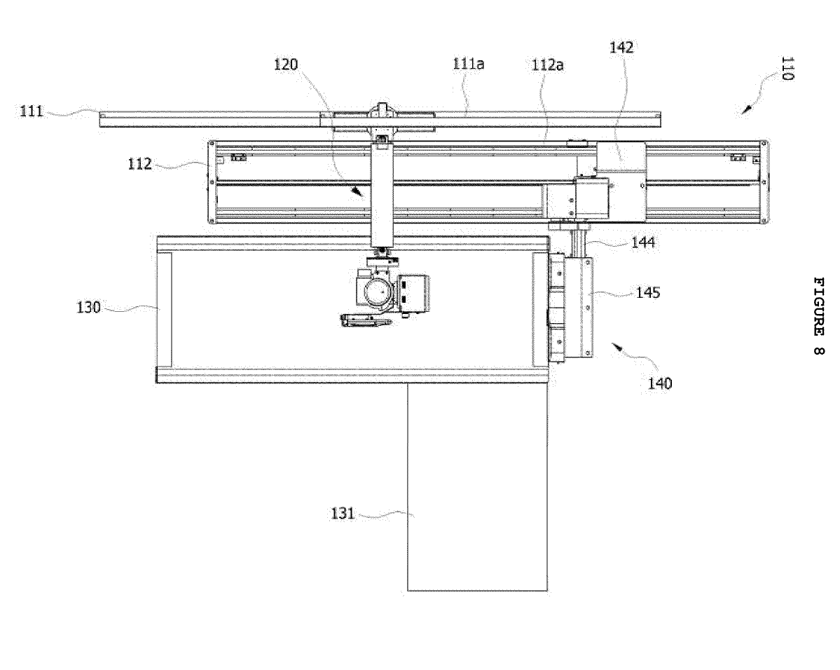

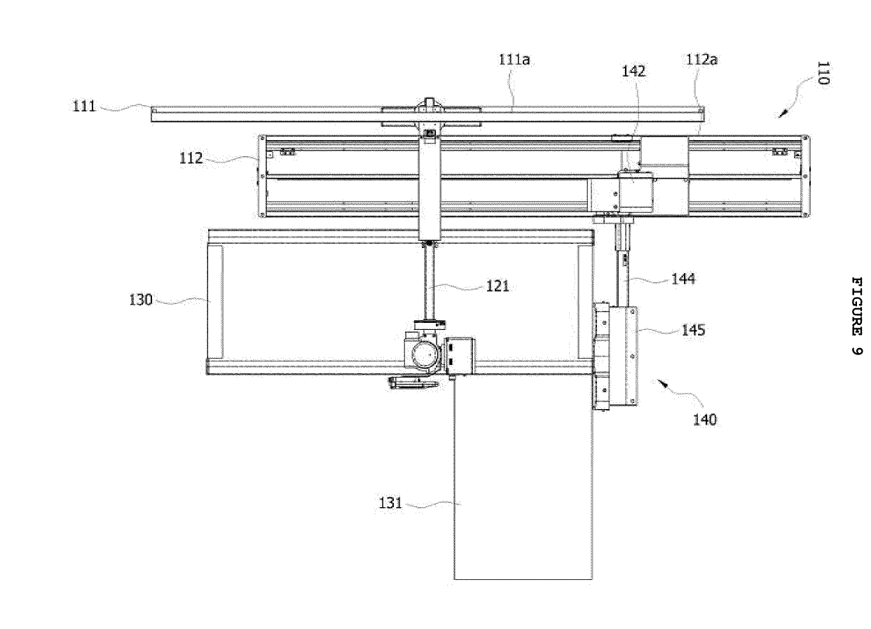

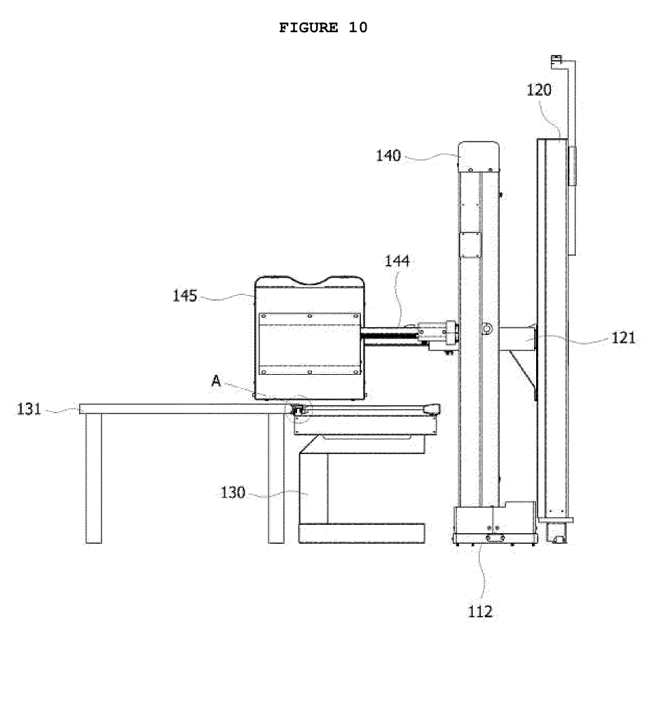

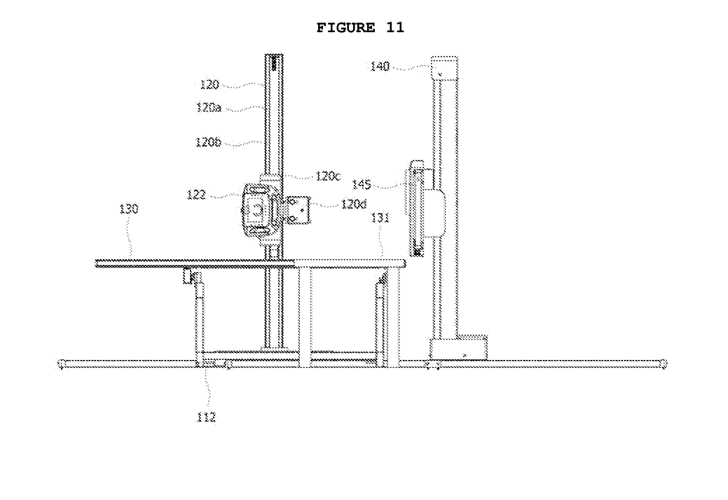

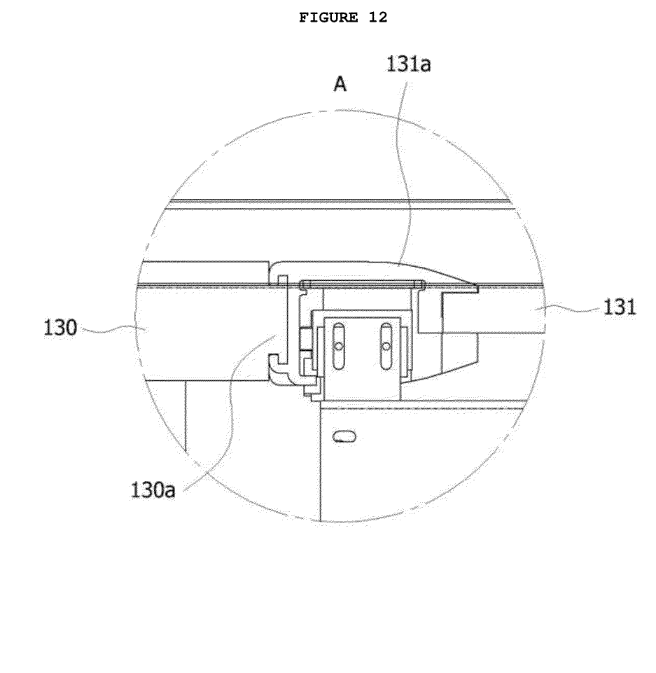

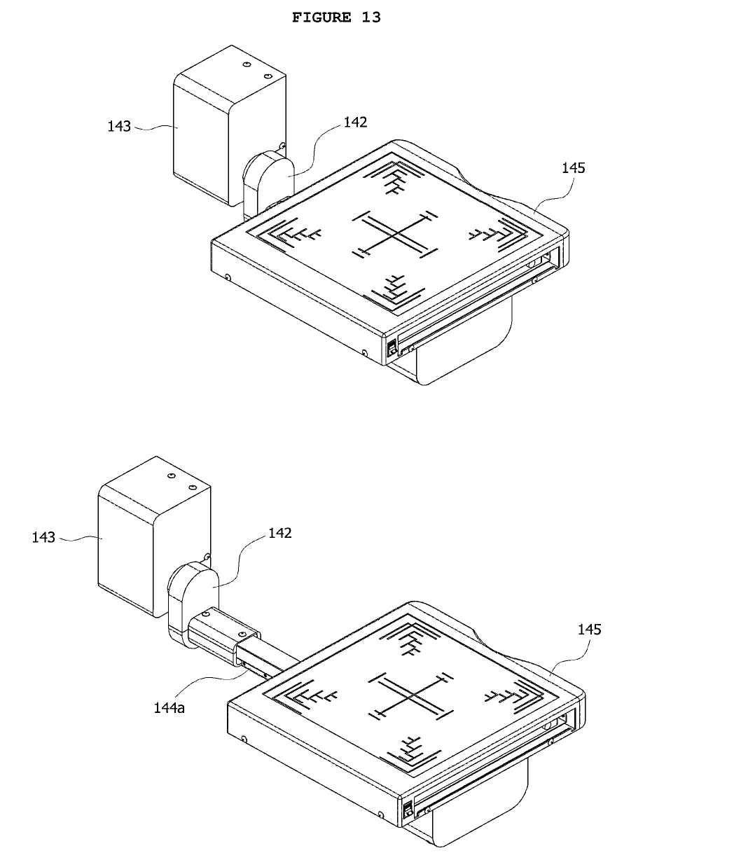

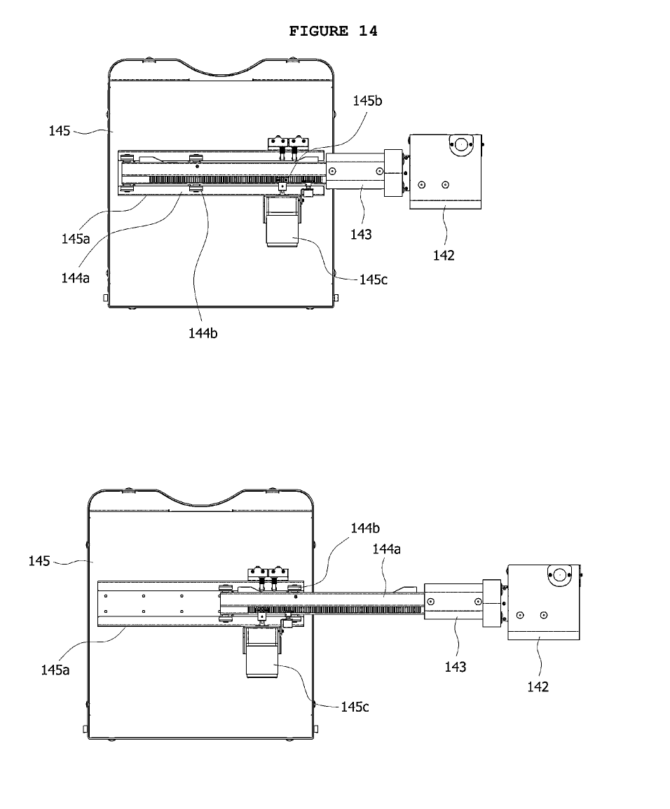

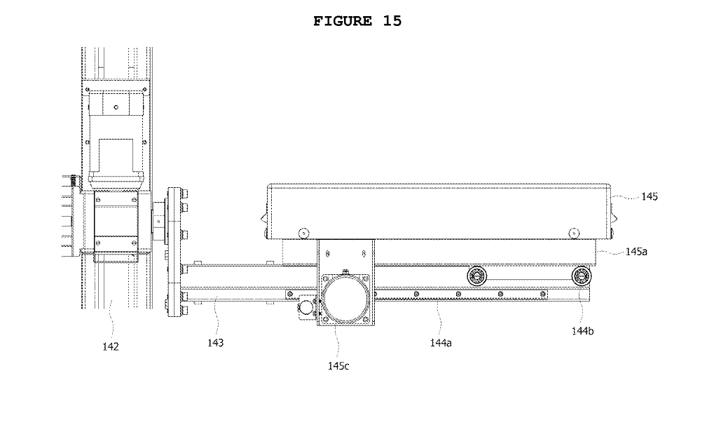

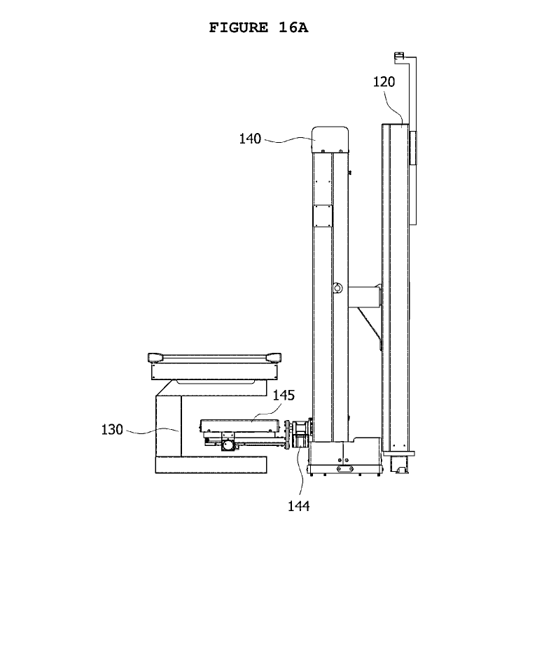

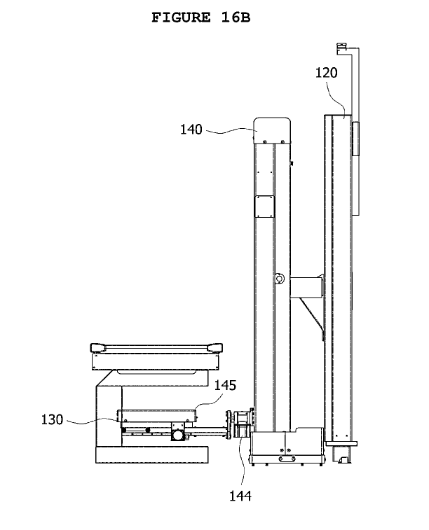

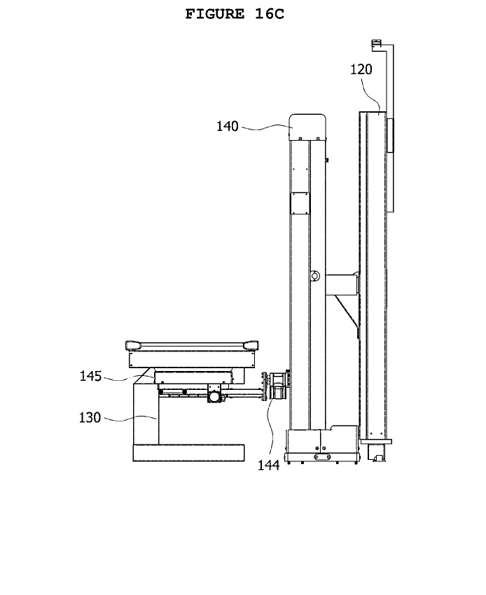

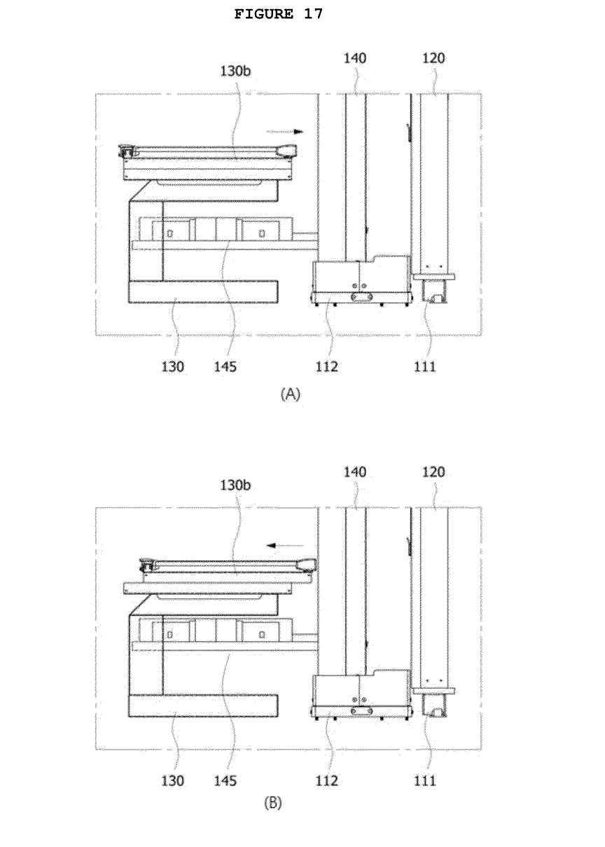

In the accompanying drawings, FIG. 2 is an exemplary view showing an example of chest photographing using a rail system including an adjustable detector according to an exemplary embodiment of the present invention; FIG. 3 is an exemplary view showing another example of FIG. 2; FIG. 4 is an exemplary view showing a further example of FIG. 3; FIG. 5A and FIG. 5B are views sequentially showing an operation in which an X-ray detector is introduced into an X-ray photography table; FIG. 6 is a view showing the state in which the X-ray detector shown in FIG. 5 has moved upwards; FIG. 7 is a view showing the state in which the X-ray detector has been introduced into the X-ray photography table; FIG. 8 and FIG. 9 are top plan views sequentially showing the operating process of the X-ray detector and an X-ray generator during Decubitus photographing using the rail system including an adjustable detector according to an exemplary embodiment of the present invention; FIG. 10 is a side elevation view of FIG. 9; FIG. 11 is a front elevation view of FIG. 9; FIG. 12 is an enlarged view of the part A shown in FIG. 10; FIG. 13 is an exemplary view illustrating that the X-ray detector shown in FIG. 7 expands in a horizontal direction; FIG. 14 is an exemplary view viewed from the opposite side to FIG. 13; FIG. 15 is an enlarged view of the X-ray detector; FIG. 16A to FIG. 16C are views sequentially illustrating the operating process of the X-ray detector which is introduced into a hollow space inside the X-ray photography table; and FIG. 17A and FIG. 17B are side exemplary views illustrating the process in which the upper section of the X-ray photography table is moved in a horizontal direction.

As shown in FIG. 2 to FIG. 7, the rail system 100 including an adjustable detector according to an exemplary embodiment of the present invention includes a rail unit 110, an X-ray tube stand 120, an X-ray photography table 130 and an X-ray sensor stand 140.

In addition, referring to FIG. 8 to FIG. 12, the rail system 100 further includes an auxiliary photography table 131 which is oriented perpendicular to the X-ray photography table 130. The auxiliary photography table 131 slides to be attached to and detached from the X-ray photography table 130.

The X-ray photography table 130 has a protrusion 130a at the portion where the X-ray photography table 130 is fastened with the auxiliary photography table 131. The auxiliary photography table 131 has a fastening portion 131a which is slidably fastened with the protrusion 130a.

The auxiliary photography table 131 is used when photographing the Decubitus posture of a patient.

In addition, the auxiliary photography table 131 can be fabricated such that it can be folded. Thus, it is possible to store the auxiliary photography table 131 in a folded shape by detaching it from the X-ray photograph table 130 that is C-shaped.

The X-ray photography table 130 can further include a foldable platform (not shown) which can be accommodated inside the X-ray photography table 130. With the foldable platform, the patient can easily lie on the X-ray photography table 130.

More specifically, the rail unit 110 is elongated in the longitudinal direction, and includes first and second rails 111 and 112 which are spaced apart from each other in a horizontal direction and are fixed to the ground.

The rail unit 110 includes, together with the first and second rails 111 and 112, first and second rack gears 111a and 112a.

The first rail 111 is elongated in the longitudinal direction and is fixed in that position.

The second rail 112 is elongated in the longitudinal direction, is fixed in that position, and is spaced apart from the first rail 111 in a horizontal direction.

The first rack gear 111a is fixedly disposed over the entire length of the first rail 111.

The second rack gear 112a is fixedly disposed over the entire length of the second rail 112.

The lateral movable structure is not limited to the above. It is apparent to a person skilled in the art that a movable structure including a ball bearing (not shown) and an LM guide can be used.

The X-ray tube stand 120 is movable in a lateral direction with respect to the rail unit 110, and is provided therein with a tube arm 121 which is movable in the vertical direction and an X-ray generator 122 which is rotatably disposed on one end of the tube arm 121.

The X-ray tube stand 120 includes, together with the tube arm 121 and the X-ray generator 122, a housing 120a, a guide rail 120b, a vertical movement section 120c and a rotation driving section 120d.

The guide rail 120b is disposed in the vertical direction inside the housing 120a, and has a rack gear on the inner surface which extends in the longitudinal direction.

The vertical movement section 120c moves in the vertical direction in response to the rotation of a pinion gear (not shown) which is engaged with a rack gear (not shown). In addition, a potential meter (not shown) is also provided inside the vertical movement section 120c.

The rotation driving section 120d is electrically connected with the X-ray generator 122, and serves to rotate the X-ray generator 122 to a preset angle.

The X-ray generator 122 is electrically connected with the rotation driving section 120d which is disposed on one end thereof.

In addition, the tube arm 121 serves to move the X-ray generator 122 in a horizontal direction.

The X-ray tube stand 120 can include a distance measuring sensor which serves to detect an interference between the X-ray tube stand 120 and the X-ray sensor stand 140.

Referring to FIG. 13 to FIG. 15, the X-ray sensor stand 140 is movable in a lateral direction with respect to the rail unit 110. The X-ray sensor stand 140 has an X-ray detector 145 disposed therein, and is operated to move an X-ray detector 145 into a hollow space in the lower part of the X-ray photography table 130 such that the center of the X-ray detector 145 and the center of the X-ray photography table 130 are positioned at the same coordinate point.

More specifically, the X-ray sensor stand 140 includes, together with the X-ray detector 145, a housing 141, a vertical movement section 142, a rotation driving section 143 and a detector arm 144.

The housing 141 is provided therein with a guide rail 141a which is disposed in the vertical direction and a rack gear (not shown).

The vertical movement section 142 moves in the vertical direction along the guide rail 141a.

The rotation driving section 143 is connected with the vertical movement section 142, and serves to rotate the X-ray detector 145.

The detector arm 144 is connected with the rotation driving section 143, with its length being adjustable in a horizontal direction using a potential meter 145c.

Here, the detector arm 144 includes a rack gear 144a, with one end thereof being connected with the rotation driving section 143, and a pulley 144b which is disposed on one end of the rack gear 144a such that the rack gear 144a can move in a forward-backward direction along a guide rail 145a.

The guide rail 145a is disposed on one surface of the X-ray detector 145 such that the detector arm 144 can move in the forward-backward direction.

More specifically, the X-ray detector 145 includes at least one pinion gear 144b which engages with the rack gear 144a of the detector arm 144 and the potential meter 145c which rotates the pinion gear 144b such that the rack gear 144a moves in a lateral direction along the guide rail 144a.

Here, distances to which the rack gear 144a is supposed to move depending on the number of revolutions of the pinion gear 144b are stored in the potential meter 145c, whereby a user can move the rack gear 144a by an intended distance.

For reference, the X-ray sensor stand 140 can further include an alarm (not shown) which generates an alarm sound when the center of the X-ray detector 145 and the center of the X-ray photography table 130 are positioned at the same point.

In addition, referring to FIG. 17A and FIG. 17B, the X-ray photography table 130 can have a slide structure. Specifically, after the X-detector 145 is introduced into the C-shaped body of the X-ray photography table 130, an upper plate 130b of the X-ray photography table 130 can move in a horizontal direction, for example, in the direction toward the X-ray tube stand 120. It is advantageous in that the upper plate 130b of the X-ray photography table 130 can move to the center of the X-ray generator depending on the size of a patient.

As set forth above, the rail system including an adjustable detector according to the present invention, which is a surgical X-ray diagnostic device, is intended to overcome the problem of requiring a long time to change the position of a patient for photographing. It is also possible to overcome the problem in that it is difficult to move the mobile table having wheels depending on the weight of the patient when changing the photographing position of the patient. The present invention has advantages in that the positions of the detector stand and the photographing position as well as the X-ray photographing position can be changed depending on the patient and a length of time required for changing the photographing position can be reduced.

In addition, in the rail system including an adjustable detector according to the present invention, it is possible to move the X-ray detector into the hollow space inside the C-shaped X-ray photography table, whereby a radiologist can take X-ray photographs of a patient at intended directions without requiring the patient to change his/her posture. It is also advantageous in that the rail system including an adjustable detector according to the present invention can be installed inside a room of a hospital that has a low floor height.

Although the foregoing descriptions of the specific exemplary embodiments of the present invention have been presented for the purposes of illustration and description, they are not intended to be exhaustive or to limit the present invention to the precise forms disclosed. Various modifications and changes are possible to a person skilled in the art that without departing from the scope of the present invention. It should not be understood that these modifications and changes can be interpreted separate from the technical principle or scope of the present invention.

What is claimed is:

1. A rail system including an adjustable detector, comprising:
a rail unit elongated in a longitudinal direction, wherein the rail unit includes first and second rails which are spaced apart from each other in a horizontal direction and are fixed to a ground;
an X-ray tube stand movable in a lateral direction with respect to the rail unit, wherein the X-ray tube stand is provided therein with a tube arm which is movable in a vertical direction and an X-ray generator which is rotatably disposed on one end of the tube arm;
a C-shaped X-ray photography table spaced apart from the rail unit; and
an X-ray sensor stand movable in a lateral direction with respect to the rail unit, wherein the X-ray sensor stand includes an X-ray detector disposed therein, and moves the X-ray detector into a hollow space in a lower part of the X-ray photography table such that a center of the X-ray detector and a center of the X-ray photography table are positioned at a same point.

2. The rail system according to claim 1, further comprising an auxiliary photography table oriented perpendicular to the X-ray photography table, wherein the auxiliary photography table is attached to and detached from the X-ray photography table by sliding, and is used when photographing a Decubitus posture of a patient.

3. The rail system according to claim 1, wherein the rail unit comprises:
a fixed first rail elongated in a longitudinal direction;
a fixed second rail elongated in the longitudinal direction, the second rail being spaced apart from the first rail in a horizontal direction;
a fixed first rack gear disposed over an entire length of the first rail; and
a fixed second rack gear disposed over an entire length of the second rail.

4. The rail system according to claim 1, wherein the X-ray sensor stand comprises:
a housing having a guide rail therein;
a vertical movement section which moves in a vertical direction along the guide rail;
a rotation driving section connected with the vertical movement section, wherein the rotation driving section rotates the X-ray detector;
a detector arm connected with the rotation driving section, a length of the detector arm being adjustable in a horizontal direction using a potential meter; and
the X-ray detector having a guide rail on one surface thereof such that the detector arm is movable in a forward-backward direction.

5. The rail system according to claim 4, wherein the detector arm comprises:
a rack gear, with one end thereof being connected with the rotation driving section; and
a pulley disposed on one end of the rack gear such that the rack gear is movable in a forward-backward direction along the guide rail.

6. The rail system according to claim 5, wherein the X-ray detector comprises:
at least one pinion gear engaging with the rack gear of the detector arm; and
a potential meter which rotates the pinion gear such that the rack gear moves along the guide rail in a lateral direction.

7. The rail system according to claim 6, further comprising a sound unit which generates an alarm sound when the center of the X-ray detector and the center of the X-ray photography table are positioned at the same point.

8. The rail system according to claim 1, wherein the X-ray tube stand further comprises a distance measuring sensor which detects an interference between the X-ray tube stand and the X-ray sensor stand.

9. The rail system according to claim 1, wherein the X-ray photography table comprises an upper plate which is expandable in a horizontal direction, whereby a center of the upper plate is freely movable to the center of the X-ray detector.

10. The rail system according to claim 2, wherein the rail unit comprises:
a fixed first rail elongated in a longitudinal direction;
a fixed second rail elongated in the longitudinal direction, the second rail being spaced apart from the first rail in a horizontal direction;
a fixed first rack gear disposed over an entire length of the first rail; and
a fixed second rack gear disposed over an entire length of the second rail.
